(19) United States
(12) Patent Application Publication
(10) Pub. No.: US 2025/0075273 A1
(43) Pub. Date: Mar. 6, 2025

(54) METHOD OF DETECTING CANCER USING GENOME-WIDE CFDNA FRAGMENTATION PROFILES

(71) Applicant: Delfi Diagnostics, Inc., Baltimore, MD (US)

(72) Inventors: Nicholas C. Dracopoli, Baltimore, MD (US); Alessandro Leal, Baltimore, MD (US); Jacob Carey, Baltimore, MD (US)

(21) Appl. No.: 18/286,081

(22) PCT Filed: Apr. 7, 2022
(86) PCT No.: PCT/US2022/023907

§ 371 Date: Oct. 6, 2023

Related U.S. Application Data
(60) Provisional application No. 63/172,493, filed on Apr. 8, 2021.

(51) Int. Cl. C12Q 1/6886; G16B 30/00

(52) U.S. Cl. CPC C12Q 1/6886; G16B 30/00

(57) ABSTRACT

The present disclosure provides methods and systems that utilize analysis of cell-free DNA (cfDNA) fragments in a sample obtained from a patient to diagnose and predict cancer status. The disclosure provides a method of detecting cancer in a subject. The disclosure also provides a method of determining overall survival of a subject having cancer. The disclosure further provides a method of monitoring cancer in a subject. Also provided are systems for genetic analysis.

### CROSS-REFERENCE TO RELATED APPLICATION(S)

This application claims benefit of priority under 35 U.S.C. § 119 (e) of U.S. Provisional Patent Application Ser. No. 63/172,493, filed Apr. 8, 2021. The disclosure of the prior application is considered part of and is incorporated by reference in the disclosure of this application.

### BACKGROUND OF THE INVENTION

### Field of the Invention

The invention relates generally to genetic analysis and more specifically to a method and system for analysis of cell-free DNA (cfDNA) fragments to detect cancer in a subject and/or assess overall survival of the subject.

### Background Information

Much of the morbidity and mortality of human cancers world-wide is a result of the late diagnosis of these diseases, where treatments are less effective. Unfortunately, clinically proven biomarkers that can be used to broadly diagnose and treat patients with early cancer are not widely available.

Analyses of cell-free DNA (cfDNA) suggests that such approaches may provide new avenues for early diagnosis and treatment. Circulating tumor DNA (ctDNA) fragments have been shown to be on average shorter than other cfDNA from non-tumor cells. Previous work has explored separating fragments into groups of different sizes caused by binding to histone core or linker proteins (e.g., short and long, or mutually exclusive sets of sizes) and using counts of these fragments to quantify ctDNA and/or classify individual samples as having presence/absence of tumor. However, previous studies have been lacking the ability to determine overall survival of a patient diagnosed with cancer, as well as providing robust sensitivity and specific in cancer detection.

### SUMMARY OF THE INVENTION

The present disclosure provides methods and systems that utilize analysis of cfDNA to detect and predict overall survival of a subject by scoring a cfDNA fragmentation profile obtained by analysis of cfDNA fragments in a sample obtained from the subject. The scoring methodology provides a measure of the overall survivability of the subject.

As such, in one embodiment, the present invention provides a method of detecting cancer in a subject. The method includes:
a) determining a cell-free DNA (cfDNA) fragmentation profile of a sample from the subject, the cfDNA fragmentation profile being determined by:
obtaining and isolating cfDNA fragments from the subject,
sequencing the cfDNA fragments to obtain sequenced fragments,
mapping the sequenced fragments to a genome to obtain windows of mapped sequences, and
analyzing the windows of mapped sequences to determine cfDNA fragment lengths and generate the cfDNA fragmentation profile; and
b) classifying the subject as having cancer or not having cancer by calculating a score based on the cfDNA fragmentation profile, the score being indicative of a likelihood of presence of cancer in the subject, thereby detecting cancer in the subject. In some aspects, the cancer excludes lung cancer. In some aspects, a chemotherapeutic agent, radiation, immunotherapy or other therapeutic regimen is administered to the subject.

In some aspects, calculating the score includes: i) determining a ratio of short to long cfDNA fragments, ii) determining a Z-score for the cfDNA fragments by chromosome arm, iii) quantifying cfDNA fragment density using a computational mixture model analysis, and iv) using a machine learning model to process output of i)-iii) to define the score.

In another embodiment, the present invention provides a method of determining overall survival of a subject having cancer. The method includes:
a) determining a cell-free DNA (cfDNA) fragmentation profile of a sample from the subject;
b) calculating a score based on the cfDNA fragmentation profile, wherein calculating the score comprises: i) determining a ratio of short to long cfDNA fragments of the sample, ii) determining a Z-score for cfDNA fragments of the sample by chromosome arm, iii) quantifying cfDNA fragment density using a computational mixture model analysis, and iv) using a machine learning model to process output of i)-iii) to define the score; and
c) determining a likelihood of overall survival of the subject based on the score, thereby determining overall survival of the subject.

In yet another aspect, the present invention provides a method of treating a subject having cancer. The method includes:
a) detecting cancer in the subject using the methodology of the invention, or determining overall survival of the subject using the methodology of the invention; and
b) administering a cancer treatment to the subject, thereby treating the subject. In some aspects, a chemotherapeutic agent, radiation, immunotherapy or other therapeutic regimen is administered to the subject.

In still another embodiment, the present invention provides a method of monitoring cancer in a subject. The method includes:
a) detecting cancer in the subject using the methodology of the invention, and/or determining overall survival of the subject using the methodology of the invention;
b) administering a cancer treatment to the subject; and
c) determining overall survival of the subject using the methodology of the invention after the cancer treatment is administered, thereby monitoring cancer in the subject. In some aspects, a chemotherapeutic agent, radiation, immunotherapy or other therapeutic regimen is administered to the subject.

In another embodiment, the invention provides a non-transitory computer readable storage medium encoded with a computer program. The computer program includes instructions that when executed by one or more processors cause the one or more processors to perform operations to perform a method of the invention.

In yet another embodiment, the invention provides a computing system. The system includes a memory, and one or more processors coupled to the memory, with the one or more processors being configured to perform operations that implement a method of the invention.

In yet another embodiment, the invention provides a system for genetic analysis and assessing cancer that includes: (a) a sequencer configured to generate a whole genome sequencing (WGS) data set for a sample; and (b) a non-transitory computer readable storage medium and/or a computer system of the invention.

### BRIEF DESCRIPTION OF THE FIGURES

FIG. 1 is a schematic diagram illustrating an exemplary DELFI approach using the methodology of the disclosure in one embodiment of the invention. Blood is collected from a cohort of healthy individuals and patients with cancer. cfDNA is extracted from the plasma fraction, processed into sequencing libraries, examined through whole genome sequencing, mapped to the genome, and analyzed to determine cfDNA fragmentation profiles across the genome. Machine learning approaches are used to generate a DELFI score and to classify individuals as healthy or as having cancer.

FIG. 2 is a table showing the performance of a cfDNA fragmentation assay for noninvasive detection of cancer. Within 3 months of inclusion, 74 patients were diagnosed with 1 of 16 different solid cancers while 207 patients did not have cancer.

FIG. 3 is a graphical plot showing data generated using the methodology of the disclosure in one embodiment of the invention. The graph shows the overall performance of a cfDNA fragmentation assay for cancer detection.

FIG. 4 is a graphical plot showing data generated using the methodology of the disclosure in one embodiment of the invention. The graph shows survival of subjects as correlated with DELFI score. Higher DELFI scores were associated with a decreased overall survival, independent of cancer stage or other clinical characteristics.

FIG. 5 is a series of graphical plots showing data curves generated using the methodology of the disclosure in one embodiment of the invention. The calculated DELFI score separates the depicted Kaplan-Meier curves of individuals with cancer (excluding lung cancer) regardless of the cutoff value used to define a high score (>0.5) versus a low score (<0.5). The number at the top of each panel indicates the determined cutoff value.

FIG. 6 is a graphical plot showing data generated using the methodology of the disclosure in one embodiment of the invention. FIG. 6 shows the results of a cox proportional hazards model in two settings. In the first setting (left panel of the plot), the DELFI score is treated as continuous. In the second setting (right panel of the plot) the DELFI score is treated as either high (>0.5) or low (<0.5). In either setting, the DELFI score is a strong predictor of survival even when adjusting for age at blood draw and stage. Note that the stage is relative to stage 1.

### DETAILED DESCRIPTION OF THE INVENTION

Described herein is a non-invasive method for the early detection of cancer, as well as prediction of overall survival of a subject having cancer. cfDNA in the blood can provide a non-invasive diagnostic avenue for patients with cancer. As demonstrated herein, DNA Evaluation of Fragments for early Interception (DELFI) was used to evaluate genome-wide fragmentation patterns of cfDNA of patients with various types of cancers, as well as healthy individuals. Evaluation of cfDNA included a scoring methodology. A defined score (also referred to herein as ‘DELFI score’) was determined for cfDNA fragmentation profiles obtained using cfDNA fragments of a given patient sample which was correlated with overall survival. Assessing cfDNA using the methodology described herein can provide a screening approach for early detection and assessment of cancer, which can increase the chance for successful treatment of a patient having cancer. Assessing cfDNA can also provide an approach for monitoring cancer, which can increase the chance for successful treatment and improved outcome of a patient having cancer.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present disclosure provides innovative methods and systems for analysis of cfDNA to detect or otherwise assess cancer. As indicated in prior studies, on average, cancer-free individuals have longer cfDNA fragments (average size of 167.09 bp) whereas individuals with cancer have shorter cfDNA fragments (average size of 164.88 bp). The methodology described herein allows simultaneous analysis of a large number of abnormalities in cfDNA through genome-wide analysis of cfDNA fragmentation patterns.

As such, in one embodiment, the present invention provides a method of detecting cancer in a subject. The method includes:
a) determining a cell-free DNA (cfDNA) fragmentation profile of a sample from the subject; and
b) classifying the subject as having cancer or not having cancer by calculating a score based on the cfDNA fragmentation profile, the score being indicative of a likelihood of presence of cancer in the subject, with the proviso that the cancer does not include lung cancer, thereby detecting cancer in the subject.

In another embodiment, the present invention provides a method of determining overall survival of a subject having cancer. The method includes:
a) determining a cell-free DNA (cfDNA) fragmentation profile of a sample from the subject;
b) calculating a score based on the cfDNA fragmentation profile, wherein calculating the score includes: i) determining a ratio of short to long cfDNA fragments of the sample, ii) determining a Z-score for cfDNA fragments of the sample by chromosome arm, iii) quantifying cfDNA fragment density using a computational mixture model analysis, and iv) using a machine learning model to process output of i)-iii) to define the score; and
c) determining a likelihood of overall survival of the subject based on the score, thereby determining overall survival of the subject.

In embodiment, the present invention provides a method of treating a subject having cancer. The method includes:
a) detecting cancer in the subject using the methodology of the invention, or determining overall survival of the subject using the methodology of the invention; and
b) administering a cancer treatment to the subject, thereby treating the subject. In some aspects, a chemotherapeutic agent, radiation, immunotherapy or other therapeutic regimen is administered to the subject.

In another embodiment, the present invention provides a method of monitoring cancer in a subject. The method includes:
a) detecting cancer in the subject using the methodology of the invention, or determining overall survival of the subject using the methodology of the invention;
b) administering a cancer treatment to the subject; and
c) determining overall survival of the subject using the methodology of the invention after the cancer treatment is administered, thereby monitoring cancer in the subject.

The methodology described herein utilizes cfDNA fragmentation profiles. As used herein, the terms “fragmentation profile,” In some aspects, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome (e.g., in non-overlapping windows) and assessed to determine a cfDNA fragmentation profile. A cfDNA fragmentation profile of a mammal having cancer is more heterogeneous (e.g., in fragment lengths) than a cfDNA fragmentation profile of a healthy mammal (e.g., a mammal not having cancer).

A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, fragment size density, median fragment size, fragment size distribution, ratio of small cfDNA fragments to large cfDNA fragments, and the coverage of cfDNA fragments. In some aspects, a cfDNA fragmentation profile can be a genome-wide cfDNA profile (e.g., a genome-wide cfDNA profile in windows across the genome). In some aspects, a cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2 q, 4 p, 5 p, 6 q, 7 p, 8 q, 9 q, 10 q, 11 q, 12 q, and/or 14 q) and a chromosomal arm (e.g., a chromosomal arm of 8 q, 13 q, 11 q, and/or 3 p). In some cases, a cfDNA fragmentation profile can include two or more targeted region profiles.

In various aspects, cfDNA obtained from a sample is isolated and fragments of a particular size range are utilized in analysis. In some aspects, analyzing excludes fragment sizes less than about 10, 50, 100 or 105 bp and greater than about 220, 250, 300, 350 bp or more. In some aspects, analyzing excludes fragment sizes less than 105 bp and greater than 170 bp. In some aspects, analyzing excludes fragment sizes less than about 230, 240, 250, 260 bp and greater than about 420, 430, 440, 450 bp or greater. In some aspects, analyzing excludes fragment sizes less than 260 bp and greater than 440 bp.

In some aspects, a cfDNA fragmentation profile may be being determined by: processing a sample from the subject comprising cfDNA fragments into sequencing libraries; subjecting the sequencing libraries to low-coverage whole genome sequencing to obtain sequenced fragments; mapping the sequenced fragments to a genome to obtain windows of mapped sequences; and analyzing the windows of mapped sequences to determine cfDNA fragment lengths.

In some aspects, a cfDNA fragmentation profile may be being determined by: obtaining and isolating cfDNA fragments from the subject, sequencing the cfDNA fragments to obtain sequenced fragments, mapping the sequenced fragments to a genome to obtain windows of mapped sequences, and analyzing the windows of mapped sequences to determine cfDNA fragment lengths and generate the cfDNA fragmentation profile.

The methodology of the present invention is based on low coverage whole genome sequencing and analysis of isolated cfDNA. In one aspect, the data used to develop the methodology of the invention is based on shallow whole genome sequence data (1-2× coverage).

In some aspects, mapped sequences are analyzed in non-overlapping windows covering the genome. Conceptually, windows may range in size from thousands to millions of bases, resulting in hundreds to thousands of windows in the genome. 5 Mb windows were used for evaluating cfDNA fragmentation patterns as these would provide over 20,000 reads per window even at a limited amount of 1-2×genome coverage. Within each window, the coverage and size distribution of cfDNA fragments was examined. In some aspects, the genome-wide pattern from an individual can be compared to reference populations to determine if the pattern is likely healthy or cancer-derived.

In certain aspects, the mapped sequences include tens to thousands of genomic windows, such as 10, 50, 100 to 1,000, 5,000, 10,000 or more windows. Such windows may be non-overlapping or overlapping and include about 1, 2, 3, 4, 5, 6, 7, 8, 9 or 10 million base pairs.

In various aspects, a cfDNA fragmentation profile is determined within each window. As such, the invention provides methods for determining a cfDNA fragmentation profile in a subject (e.g., in a sample obtained from a subject).

In some aspects, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some aspects, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

In various aspects, a cfDNA fragmentation profile can include a cfDNA fragment size pattern. cfDNA fragments can be any appropriate size. For example, in some aspects, a cfDNA fragment can be from about 50 base pairs (bp) to about 400 bp in length. As described herein, a subject having cancer can have a cfDNA fragment size pattern that contains a shorter median cfDNA fragment size than the median cfDNA fragment size in a healthy subject. A healthy subject (e.g., a subject not having cancer) can have cfDNA fragment sizes having a median cfDNA fragment size from about 166.6 bp to about 167.2 bp (e.g., about 166.9 bp). In some aspects, a subject having cancer can have cfDNA fragment sizes that are, on average, about 1.28 bp to about 2.49 bp (e.g., about 1.88 bp) shorter than cfDNA fragment sizes in a healthy subject. For example, a subject having cancer can have cfDNA fragment sizes having a median cfDNA fragment size of about 164.11 bp to about 165.92 bp (e.g., about 165.02 bp).

In some aspects, a dinucleosomal cfDNA fragment can be from about 230 base pairs (bp) to about 450 bp in length. As described herein, a subject having cancer can have a dinucleosomal cfDNA fragment size pattern that contains a shorter median dinucleosomal cfDNA fragment size than the median dinucleosomal cfDNA fragment size in a healthy subject. In some aspects, on average, cancer-free subjects have longer cfDNA fragments in the dinucleosomal range (average size of 334.75 bp) whereas subjects with cancer have shorter dinucleosomal cfDNA fragments (average size of 329.6 bp). As such, a healthy subject (e.g., a subject not having cancer) can have dinucleosomal cfDNA fragment sizes having a median cfDNA fragment size of about 334.75 bp. In some aspects, a subject having cancer can have dinucleosomal cfDNA fragment sizes that are shorter than dinucleosomal cfDNA fragment sizes in a healthy subject. For example, a subject having cancer can have dinucleosomal cfDNA fragment sizes having a median cfDNA fragment size of about 329.6 bp.

A cfDNA fragmentation profile can include a cfDNA fragment size distribution. As described herein, a subject having cancer can have a cfDNA size distribution that is more variable than a cfDNA fragment size distribution in a healthy subject. In some aspects, a size distribution can be within a targeted region. A healthy subject (e.g., a subject not having cancer) can have a targeted region cfDNA fragment size distribution of about 1 or less than about 1. In some aspects, a subject having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy subject. In some aspects, a subject having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy subject. In some aspects, a subject having cancer can have a targeted region cfDNA fragment size distribution that is about 47 bp smaller to about 30 bp longer than a targeted region cfDNA fragment size distribution in a healthy subject. In some aspects, a subject having cancer can have a targeted region cfDNA fragment size distribution of, on average, a 10, 11, 12, 13, 14, 15, 15, 17, 18, 19, 20 or more bp difference in lengths of cfDNA fragments. For example, a subject having cancer can have a targeted region cfDNA fragment size distribution of, on average, about a 13 bp difference in lengths of cfDNA fragments. In some aspects, a size distribution can be a genome-wide size distribution.

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. As described herein, a subject having cancer can have a correlation of fragment ratios (e.g., a correlation of cfDNA fragment ratios to reference DNA fragment ratios such as DNA fragment ratios from one or more healthy subjects) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy subject. A healthy subject (e.g., a subject not having cancer) can have a correlation of fragment ratios (e.g., a correlation of cfDNA fragment ratios to reference DNA fragment ratios such as DNA fragment ratios from one or more healthy subjects) of about 1 (e.g., about 0.96). In some aspects, a subject having cancer can have a correlation of fragment ratios (e.g., a correlation of cfDNA fragment ratios to reference DNA fragment ratios such as DNA fragment ratios from one or more healthy subjects) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy subjects) in a healthy subject.

The methodology of the present invention further includes calculating a score (e.g., DELFI score) based on a cfDNA fragmentation profile. In some aspects, calculating the score includes: i) determining a ratio of short to long cfDNA fragments of the sample, ii) determining a Z-score for cfDNA fragments of the sample by chromosome arm, iii) quantifying cfDNA fragment density using a computational mixture model analysis, and iv) using a machine learning model to process output of i)-iii) to define the score. In various aspects, the score is utilized to determine a likelihood of overall survival of the subject.

In one illustrative example (Example 1), in a multi-cancer cohort, the inventors calculated from low coverage whole genome sequencing the ratio of short to long fragments by 5 MB bins, Z-scores by chromosome arm, and a mixture model of cfDNA fragment sizes, for each individual. Using these features as input, the inventors fit a cross-validated gradient boosted machine to the cancer status of each person (Cancer/No Cancer). The output of this model is a score ranging from 0 to 1, with high numbers indicating a stronger signal of cancer and low numbers more similarity to non-cancer. Once complete, only the samples with a diagnosis of cancer are retained.

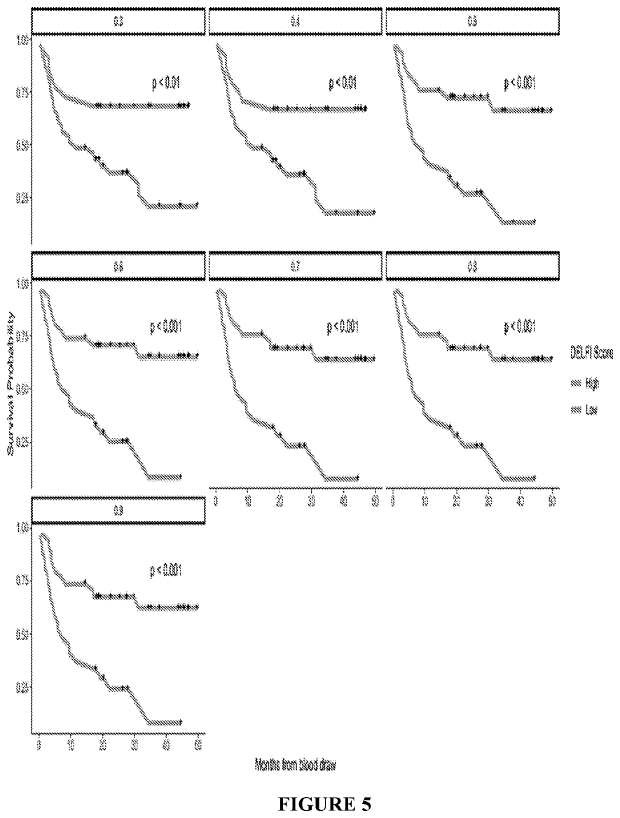

In some aspects, the outputted score is analyzed as follows. Using follow-up time, whether or not the patient is alive at the end of follow-up, and the score from the machine learning model above, the relationship of fragmentation of cfDNA and survival was determined. As shown in FIG. 5, strong separation in Kaplan-Meier curves with a high versus low score in individuals with cancer was determined. Additionally, the independence of this score from other clinical features was assessed by fitting a cox proportional hazards model, regressing on score, cancer stage, and patient age.

With reference to FIG. 5, as discussed above, the calculated DELFI score separates the depicted Kaplan-Meier curves of individuals with cancer (excluding lung cancer) regardless of the cutoff value used to define a high score (>0.5) versus a low score (<0.5). The number at the top of each panel indicates the determined cutoff value.

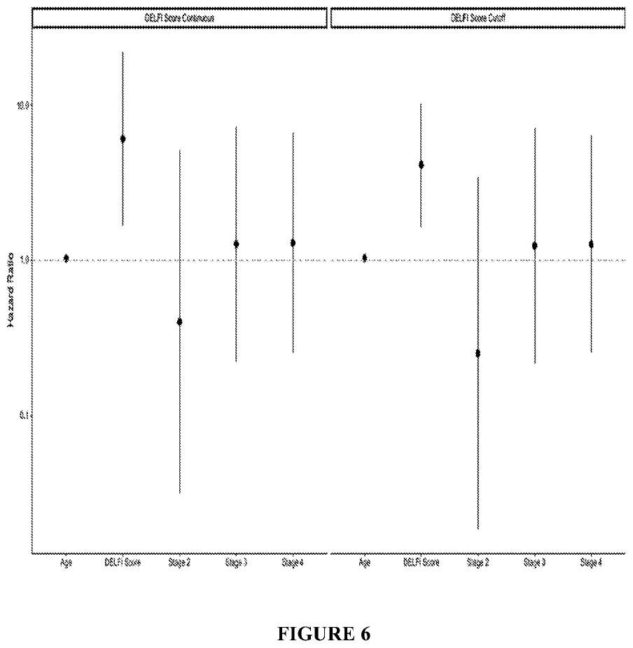

FIG. 6 shows the results of a cox proportional hazards model in two settings. In the first setting (left panel of the plot), the DELFI score is treated as continuous. In the second setting (right panel of the plot) the DELFI score is treated as either high (>0.5) or low (<0.5). In either setting, the DELFI score is a strong predictor of survival even when adjusting for age at blood draw and stage. Note that the stage is relative to stage 1.

The presently described methods and systems are useful for detecting, predicting, treating and/or monitoring cancer status in a subject. Any appropriate subject, such as a mammal can be assessed, monitored, and/or treated as described herein. Examples of some mammals that can be assessed, monitored, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein.

A subject having, or suspected of having, any appropriate type of cancer can be assessed and/or treated (e.g., by administering one or more cancer treatments to the subject) using the methods and systems described herein. A cancer can be any stage cancer. In some aspects, a cancer can be an early stage cancer. In some aspects, a cancer can be an asymptomatic cancer. In some aspects, a cancer can be a residual disease and/or a recurrence (e.g., after surgical resection and/or after cancer therapy). A cancer can be any type of cancer. Examples of types of cancers that can be assessed, monitored, and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

When treating a subject having, or suspected of having, cancer as described herein, the subject can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation, surgical intervention, adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g., the guidelines for therapy from the American Society of Clinical Oncology (ASCO), European Society for Medical Oncology (ESMO), or National Comprehensive Cancer Network (NCCN).

When monitoring a subject having, or suspected of having, cancer as described herein, the monitoring can be before, during, and/or after the course of a cancer treatment. Methods of monitoring provided herein can be used to determine the efficacy of one or more cancer treatments and/or to select a subject for increased monitoring.

In some aspects, the monitoring can include conventional techniques capable of monitoring one or more cancer treatments (e.g., the efficacy of one or more cancer treatments). In some aspects, a subject selected for increased monitoring can be administered a diagnostic test (e.g., any of the diagnostic tests disclosed herein) at an increased frequency compared to a subject that has not been selected for increased monitoring. For example, a subject selected for increased monitoring can be administered a diagnostic test at a frequency of twice daily, daily, bi-weekly, weekly, bi-monthly, monthly, quarterly, semi-annually, annually, or any at frequency therein.

In various aspects, DNA is present in a biological sample taken from a subject and used in the methodology of the invention. The biological sample can be virtually any type of biological sample that includes DNA. The biological sample is typically a fluid, such as whole blood or a portion thereof with circulating cfDNA. In embodiments, the sample includes DNA from a tumor or a liquid biopsy, such as, but not limited to amniotic fluid, aqueous humor, vitreous humor, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid. In one aspect, the sample includes DNA from a circulating tumor cell.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

The methods and systems of the disclosure utilize nucleic acid sequence information, and can therefore include any method or sequencing device for performing nucleic acid sequencing including nucleic acid amplification, polymerase chain reaction (PCR), nanopore sequencing, 454 sequencing, insertion tagged sequencing. In some aspects, the methodology or systems of the disclosure utilize systems such as those provided by Illumina, Inc, (including but not limited to HiSeq™ X10, HiSeq™ 1000, HiSeq™ 2000, HiSeq™ 2500, Genome Analyzers™, MiSeq™, NextSeq, NovaSeq 6000 systems), Applied Biosystems Life Technologies (SOLID™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II).

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Accordingly, the invention further provides a system for detecting, analyzing and/or assessing cancer. In various aspects, the system includes: (a) a sequencer configured to generate a low-coverage whole genome sequencing data set for a sample; and (b) a computer system and/or processor with functionality to perform a method of the invention.

In some aspects, the computer system further includes one or more additional modules. For example, the system may include one or more of an extraction and/or isolation unit operable to select suitable genetic components analysis, e.g., cfDNA fragments of a particular size.

In some aspects, the computer system further includes a visual display device. The visual display device may be operable to display a curve fit line, a reference curve fit line, and/or a comparison of both.

Methods for detection and analysis according to various aspects of the present invention may be implemented in any suitable manner, for example using a computer program operating on the computer system. As discussed herein, an exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The system according to various aspects of the present invention and its various elements provide functions and operations to facilitate detection and/or analysis, such as data gathering, processing, analysis, reporting and/or diagnosis. For example, in the present aspect, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of a disease status model and/or diagnosis information.

The procedures performed by the system may comprise any suitable processes to facilitate analysis and/or cancer diagnosis. In one embodiment, the system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

### Detecting Cancer Using Genome-Wide cfDNA Fragmentation in a Prospective Diagnostic Cohort

Genome-wide cfDNA fragmentation patterns have been demonstrated to distinguish with high sensitivity and specificity between plasma samples from individuals with and without cancer.

In this example, the methodology of the present disclosure was utilized to detect cancer and predict overall patient survival.

The objective of the study was to evaluate the cfDNA fragmentation assay as a blood-based screening test to detect multiple different solid tumors and predict overall patient survival by using a computational scoring scheme.

### Methods

Plasma Samples: Samples were collected from 281 patients referred to Diagnostic Outpatient Clinic of the Herlev and Gentofte Hospital (Copenhagen University Hospital, Copenhagen, Denmark) due to non-organ specific signs and symptoms of cancer.

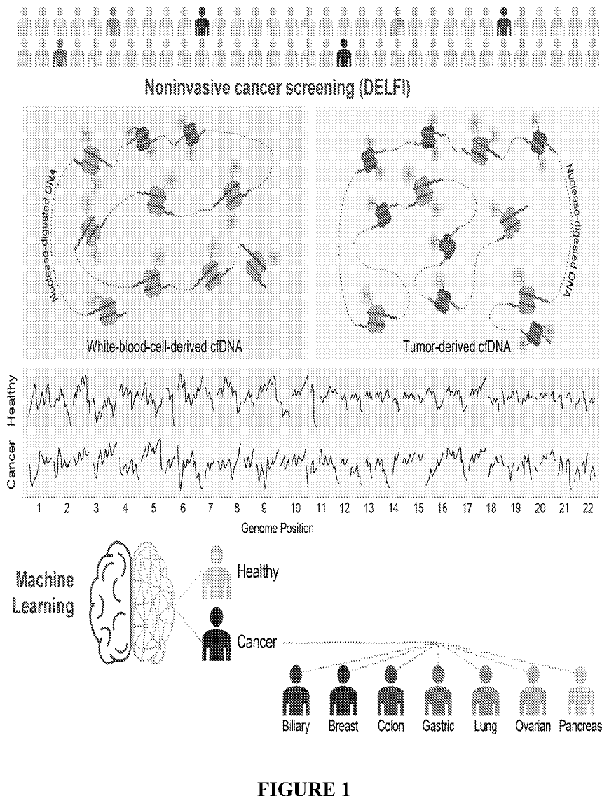

cfDNA Fragmentation Approach: The cfDNA fragmentation approach is summarized in FIG. 1. cfDNA was extracted from plasma, processed into sequencing libraries, examined by low-coverage whole-genome sequencing (WGS), mapped to the genome, and analyzed to determine cfDNA fragmentation profiles across the genome.

Machine learning was used to generate a DELFI score and to classify individuals as healthy or having cancer and predict overall patient survival.

### Results

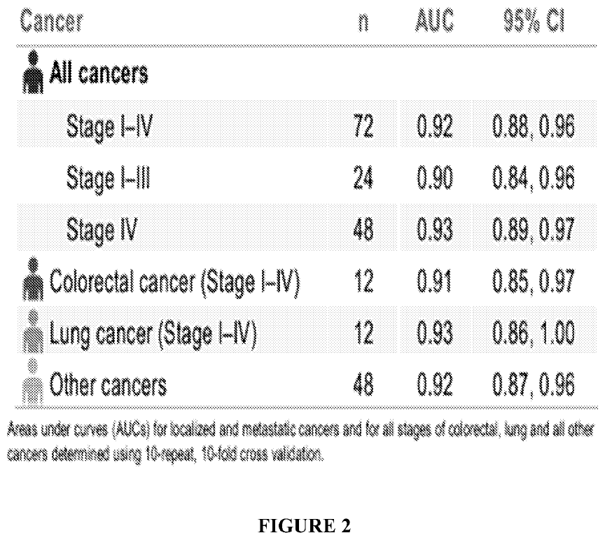

Performance of cfDNA Fragmentation Assay for Noninvasive Detection of Cancer: Within 3 months of inclusion, 74 patients were diagnosed with 1 of 16 different solid cancers while 207 patients did not have cancer. Additional results are shown in FIG. 2. Areas under curves (AUCs) for localized and metastatic cancers and for all stages of colorectal, lung and all other cancers determined using 10-repeat, 10-fold cross validation.

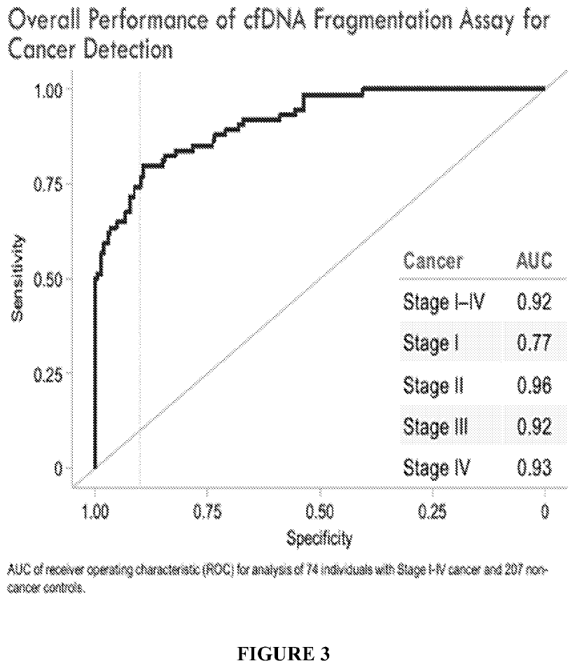

Overall Performance of cfDNA Fragmentation Assay for Cancer Detection: Results are summarized in FIG. 3. AUC of receiver operating characteristic (ROC) for analysis of 74 individuals with Stage I-IV cancer and 207 non-cancer controls.

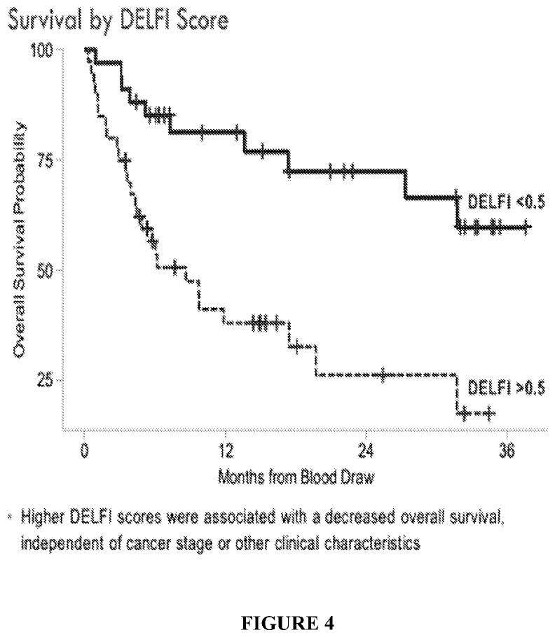

Survival by DELFI Score: Higher DELFI scores were associated with a decreased overall survival, independent of cancer stage or other clinical characteristics as shown in FIG. 4. FIG. 4 shows survival of subjects as correlated with DELFI score. Higher DELFI scores were associated with a decreased overall survival, independent of cancer stage or other clinical characteristics.

### CONCLUSION

This study of prospectively enrolled individuals demonstrated the ability of the cfDNA fragmentation assay to distinguish between individuals with and without cancer. The assay of the invention displayed high performance in a multi-cancer setting using only fragmentation-related information obtained from low-coverage WGS.

The results suggest that machine learning models can differentiate between cancer and non-cancer despite the presence of common nonmalignant conditions (including cardiovascular, autoimmune, or inflammatory diseases) using cfDNA fragmentation profiles. Additionally, individuals with higher DELFI scores had a worse prognosis, independent of other characteristics.

These data support development of genome-wide cfDNA fragmentation analyses for noninvasive detection of both single and multiple cancers.

Although the invention has been described with reference to the above examples, it will be understood that modifications and variations are encompassed within the spirit and scope of the invention. Accordingly, the invention is limited only by the following claims.

1. A method of detecting cancer in a subject, comprising:
a) determining a cell-free DNA (cfDNA) fragmentation profile of a sample from the subject, the cfDNA fragmentation profile being determined by:
obtaining and isolating cfDNA fragments from the subject,
sequencing the cfDNA fragments to obtain sequenced fragments,
mapping the sequenced fragments to a genome to obtain windows of mapped sequences, and
analyzing the windows of mapped sequences to determine cfDNA fragment lengths and generate the cfDNA fragmentation profile; and
b) classifying the subject as having cancer or not having cancer by calculating a score based on the cfDNA fragmentation profile, the score being indicative of a likelihood of presence of cancer in the subject, thereby detecting cancer in the subject.

2. The method of claim 1, wherein calculating the score comprises: i) determining a ratio of short to long cfDNA fragments, ii) determining a Z-score for the cfDNA fragments by chromosome arm, iii) quantifying cfDNA fragment density using a computational mixture model analysis, and iv) using a machine learning model to process output of i)-iii) to define the score.

3. The method of claim 2, wherein the score has a range of 0 to 1.

4-7. (canceled)

8. The method of claim 1, wherein sequencing comprises subjecting the cfDNA fragments to low coverage whole-genome sequencing to obtain the sequenced fragments.

9. The method of claim 1, wherein isolating cfDNA fragments comprises excluding fragment sizes less than 105 bp and greater than 170 bp.

10. The method of claim 1, wherein the windows of mapped sequences comprise tens to thousands of windows.

11-13. (canceled)

14. The method of claim 1, wherein the cfDNA fragmentation profile comprises a ratio of small cfDNA fragments to large cfDNA fragments in the windows of mapped sequences.

15. The method of claim 1, wherein the cfDNA fragmentation profile comprises the sequence coverage of small and large cfDNA fragments in windows across the genome.

16. The method of claim 1, wherein the cfDNA fragmentation profile is over the whole genome.

17. The method of claim 1, wherein the cfDNA fragmentation profile is over a subgenomic interval.

18. The method of claim 1, wherein classifying comprises comparing the cfDNA fragmentation profile to a reference cfDNA fragmentation.

19. (canceled)

20. The method of claim 1, wherein the cancer is a solid tumor.

21-22. (canceled)

23. The method of claim 1, wherein the cancer is a hematologic cancer.

24. (canceled)

25. The method of claim 1, further comprising administering a cancer treatment to the subject.

26. (canceled)

27. A method of determining overall survival of a subject having cancer comprising:
a) determining a cell-free DNA (cfDNA) fragmentation profile of a sample from the subject;
b) calculating a score based on the cfDNA fragmentation profile, wherein calculating the score comprises: i) determining a ratio of short to long cfDNA fragments of the sample, ii) determining a Z-score for cfDNA fragments of the sample by chromosome arm, iii) quantifying cfDNA fragment density using a computational mixture model analysis, and iv) using a machine learning model to process output of i)-iii) to define the score; and
c) determining a likelihood of overall survival of the subject based on the score, thereby determining overall survival of the subject.

28. The method of claim 27, wherein the score has a range of 0 to 1.

29-30. (canceled)

31. The method of claim 27, wherein the cfDNA fragmentation profile is determined by:
obtaining and isolating cfDNA fragments from the subject,
sequencing the cfDNA fragments to obtain sequenced fragments, mapping the sequenced fragments to a genome to obtain windows of mapped sequences, and
analyzing the windows of mapped sequences to determine cfDNA fragment lengths and generate the cfDNA fragmentation profile.

32-41. (canceled)

42. The method of claim 27, wherein the cancer is a solid tumor.

43-44. (canceled)

45. The method of claim 27, wherein the cancer is a hematologic cancer.

46. (canceled)

47. The method of claim 27, further comprising administering a cancer treatment to the subject.

48. (canceled)

49. A method of treating a subject having cancer comprising:
a) detecting cancer in the subject using the method of claim 1, and
b) administering a cancer treatment to the subject, thereby treating the subject.

50. The method of claim 49, wherein the cancer is a solid tumor.

51-52. (canceled)

53. The method of claim 49, wherein the cancer is a hematologic cancer.

54. (canceled)

55. The method of claim 49, wherein the cancer treatment is selected from the group consisting of surgery, adjuvant chemotherapy, neoadjuvant chemotherapy, radiation therapy, hormone therapy, cytotoxic therapy, immunotherapy, adoptive T cell therapy, targeted therapy, or any combinations thereof.

56. The method of claim 49, wherein the subject is a human.

57-59. (canceled)
